(19) United States
(12) Patent Application Publication
(10) Pub. No.: US 2024/0335120 A1
(43) Pub. Date: Oct. 10, 2024

(54) SYSTEMS AND METHOD TO MEASURE CORE BODY TEMPERATURE BY A VARYING GRADIENT

(71) Applicant: KONINKLIJKE PHILIPS N.V., EINDHOVEN (NL)

(72) Inventors: Warner Rudolph Theophile Ten Kate, Eindhoven (NL); Jozef Hubertus Gelissen, Eindhoven (NL)

(21) Appl. No.: 18/617,968

(22) Filed: Mar. 27, 2024

Related U.S. Application Data
(60) Provisional application No. 63/457,435, filed on Apr. 6, 2023.

(51) Int. Cl. A61B 5/01; A61B 5/00

(52) U.S. Cl. CPC A61B 5/01; A61B 5/681; A61B 5/6833; A61B 5/7278; A61B 2560/0223; A61B 2560/0252; A61B 2562/0271

(57) ABSTRACT

A core body temperature sensor (10) includes a thermoresistant structure (12) having a proximal surface (11) configured for attachment to skin of an associated patient, and a distal end (13) opposite from the proximal surface. A plurality of temperature sensors (14) is arranged to measure temperatures of the thermoresistant structure at a corresponding plurality of different distances from the proximal surface of the thermoresistant structure. A temperature-changing element (16) is configured to change a temperature of the distal end of the thermoresistant structure. An electronic processor (20) is programmed to acquire temperature measurements from the plurality of temperature sensors while operating the temperature-changing element to maintain a temperature of the distal end of the thermoresistant structure at a temperature different from an ambient temperature, and extrapolate a core body temperature of the associated patient from the acquired temperature measurements.

### CROSS-REFERENCE TO RELATED APPLICATIONS

This patent application claims the priority benefit under 35 U.S.C. § 119(e) of U.S. Provisional Application No. 63/457,435, filed on Apr. 6, 2023, the contents of which are herein incorporated by reference.

### VARYING GRADIENT

The following relates generally to the medical monitoring arts, wearable medical monitor arts, core body temperature measurement arts, and related arts.

### BACKGROUND

Health-related unobtrusive sensing systems enable replacement of continued hospitalization with obtrusive vital signs sensor technologies, centered around the individual, to provide remote monitoring of the subject's general health condition. Vital signs monitoring typically includes monitoring one or more of the following physical parameters: heart rate (HR), blood pressure (BP), respiratory rate (RR), core body temperature and blood oxygenation (SpO₂).

Core body temperature (CBT) is considered one of the vital signs to inform about a human's health state. The CBT is the temperature of deep organs such as in the torso cavity, and hence more accurately reflects health status than, for example, the temperature of exposed skin. But CBT cannot be directly measured. In a clinical setting, CBT is commonly estimated by thermometers that contact the regions of the human body that are known to be at a temperature that is close to CBT (oral, rectal or in-ear are preferred from a precision point of view). However, the use of thermometers applied to these regions is not feasible, or at least cumbersome, in an ambulatory setting in which an outpatient is expected to wear the sensor as the patient is engaged in daily activities such as walking, driving, eating, and so forth. Health patches (such as the Vital Connect patch (also known as Philips Biosensor and available from Philips BioTel) are outfitted with a temperature sensor that actually measures skin temperature. The issue with skin temperature is that it does not reflect CBT, but is reactive to it.

The human body has a heat gradient where CBT is around 36.5-37.5° C. for healthy adults, and a peripheral temperature of ˜32° C. (e.g. fingers). When the CBT tends to overshoot (e.g. from a physical activity), the peripheral temperature increases in order to dissipate heat. When CBT tends to drop too much the peripheral temperature decreases in order to diminish heat loss. Secondly, when the CBT setpoint changes (e.g. onset of flu or infection, core body temperature increases), peripheral temperature decreases in order to diminish heat loss.

While the skin temperature reacts to changes in CBT, there are numerous, potentially confounding, physiological processes. The skin is an active organ in thermoregulation. For example, vasoconstriction or vasodilation of blood vessels in the skin can occur in response to cold or heat (e.g., vasoconstriction reduces blood content of the skin and hence reduces body heat loss through the skin; while, vasodilation increases blood content of the skin). But other factors can cause such skin responses, such as blushing which is vasodilation as an emotional response. Hence, inferring CBT from a measurement of skin temperature measurement is unlikely to be accurate.

The following discloses certain improvements to overcome these problems and others.

### SUMMARY

In one aspect, a core body temperature sensor includes a thermoresistant structure having a proximal surface configured for attachment to skin of an associated patient, and a distal end opposite from the proximal surface. A plurality of temperature sensors is arranged to measure temperatures of the thermoresistant structure at a corresponding plurality of different distances from the proximal surface of the thermoresistant structure. A temperature-changing element is configured to change a temperature of the distal end of the thermoresistant structure. An electronic processor is programmed to acquire temperature measurements from the plurality of temperature sensors while operating the temperature-changing element to maintain a temperature of the distal end of the thermoresistant structure at a temperature different from an ambient temperature, and extrapolate a core body temperature of the associated patient from the acquired temperature measurements.

In another aspect, a core body temperature measurement method includes disposing a proximal surface of a thermoresistant structure in thermal contact with skin of a patient; measuring temperatures of the thermoresistant structure at a plurality of different distances from the proximal surface of the thermoresistant structure; during the measuring of the temperatures, maintaining a temperature of a distal end of the thermoresistant structure at a temperature different from an ambient temperature using a heating or cooling element; and using an electronic processor, extrapolating a core body temperature of the patient from the measured temperatures.

One advantage resides in providing an accurate CBT of a patient.

Another advantage resides in compensating for a difference in temperature between skin of a patient and a temperature sensing device attached to the patient.

Another advantage resides in compensating for local heating of a temperature measuring device attached to a patient while measuring a CBT of the patient.

Another advantage resides in accounting for thermal resistance between a temperature measuring device attached to a patient while measuring a CBT of the patient.

Another advantage resides in adding a heating or cooling element to a temperature sensing device for measuring CBT of a patient to improve accuracy of a CBT measurement.

A given embodiment may provide none, one, two, more, or all of the foregoing advantages, and/or may provide other advantages as will become apparent to one of ordinary skill in the art upon reading and understanding the present disclosure.

### BRIEF DESCRIPTION OF THE DRAWINGS

The disclosure may take form in various components and arrangements of components, and in various steps and arrangements of steps. The drawings are only for purposes of illustrating the preferred embodiments and are not to be construed as limiting the disclosure.

FIG. 1 diagrammatically shows a core body temperature sensor in accordance with the present disclosure.

FIG. 2 shows the core body temperature sensor of FIG. 1 implemented in a wristwatch.

FIG. 3 shows an example of a process performed by the sensor of FIG. 1.

### DETAILED DESCRIPTION

As used herein, the singular form of “a,” “an”, and “the” include plural references unless the context clearly dictates otherwise. As used herein, statements that two or more parts or components are “coupled,” “connected,” or “engaged” shall mean that the parts are joined, operate, or co-act together either directly or indirectly, i.e., through one or more intermediate parts or components, so long as a link occurs. Directional phrases used herein, such as, for example and without limitation, top, bottom, left, right, upper, lower, front, back, and derivatives thereof, relate to the orientation of the elements shown in the drawings and are not limiting upon the scope of the claimed invention unless expressly recited therein. The word “comprising” or “including” does not exclude the presence of elements or steps other than those described herein and/or listed in a claim. In a device comprised of several means, several of these means may be embodied by one and the same item of hardware.

As previously noted, inference of CBT from a skin temperature measurement is unlikely to be accurate. To improve accuracy, the CBT measurement can include a stack of thermoresistant foam layers, where the temperature between each layer of the stack is measured in addition to measuring the skin temperature. The different temperature readings enable extrapolation of the CBT, based on known thermal resistances (i.e., thermoresistances) of the layers of the stack. Typically, this can be done by linear extrapolation, using the ratio of the subsequent thermoresistances and the differences of the measured temperatures.

A difficulty with this approach is that a temperature difference is required between the sensed body tissue and the environment of the stack. The differences between the different layers will vanish if this end-to-end difference is small. This can happen, for example, when the sensor stack is beneath clothing, such as the sleeve of a coat or sweater, or when the sensor is attached at the torso beneath clothes. In another example, if the ambient air is the same as or close to the CBT then there will be no temperature difference even if the skin is exposed. This is particularly problematic for ambulatory CBT sensors since on a summer day with the patient outside or in an interior space with no air conditioning, the ambient temperature can easily be at or close to the CBT of 36.5-37.5° C. (97.7-99.5° F.) of a healthy adult such that a remaining temperature difference is small and no temperature gradient over the CBT sensors is present. Even if there is some CBT versus ambient temperature difference, if that difference is small then the accuracy of the CBT measurement will suffer. Another cause of diminishing temperature differences is the sensor system being part of further electronics, such as a smart watch, where the processing of the electronics introduce local heating. This local heating can also vary due to the varying computational tasks the smart-watch is executing. In embodiments disclosed herein, the temperature difference is actively raised or lowered to ensure a sufficient temperature difference over the stack to provide a CBT measurement of high accuracy.

Another difficulty resides in the thermal resistance between the sensors and the body. For example, a wearable ambulatory health patch is typically held on the skin by an adhesive, which introduces a thermal barrier (the adhesive) between the “skin” temperature sensor and the skin. The thermal resistance of the adhesive may be nonnegligible, and moreover may depend on factors such as manufacturing variability in the thickness of the adhesive (the health patch is a single-use consumable item and hence may not be manufactured to high precision) and the force used to press the health patch onto the skin (more force can spread out the adhesive and reduce its thermal resistance). A different thermal contact resistance leads to a different temperature distribution over the stack, which in turn affects the precision of the measurement. Embodiments are disclosed herein by which the thermal resistance of the adhesive or other interfacial resistance is estimated and accounted for in extrapolating the CBT measurement.

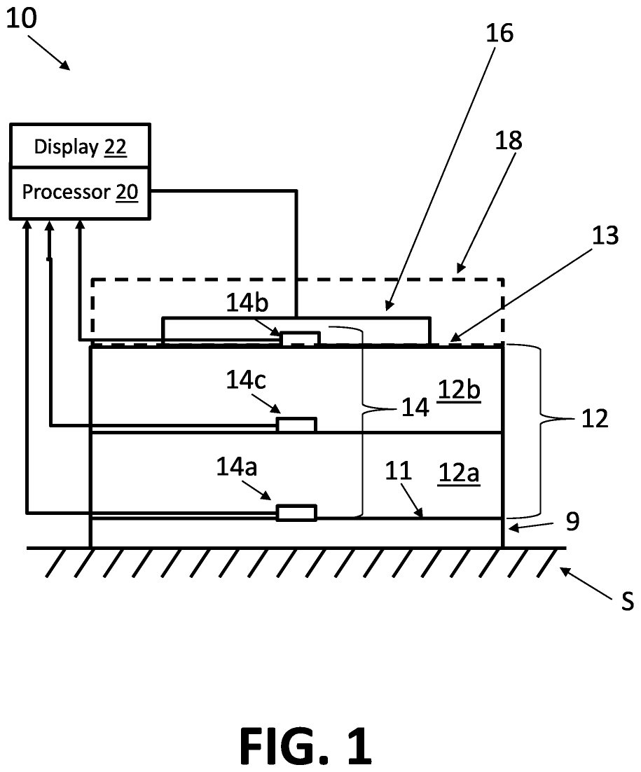

With reference to FIG. 1, a core body temperature (CBT) sensor 10 for measuring a CBT of an associated patient is shown. As used herein, the term “patient” (and variants thereof) refers to, and includes, an outpatient, a discharged patient, a patient undergoing screening using the monitoring device as part of an annual medical checkup, or other person whose health condition (including at least CBT) is to be monitored. As shown in FIG. 1, the core body temperature sensor 10 is wearable by, or otherwise attached to, skin S of the patient. The wearable monitoring device 10 can include any suitable monitoring device, such as Vital Connect patch (available from Koninklijke Philips N.V., Eindhoven, the Netherlands), or a medical wearable device, a torso-worn vital signs health patch, a wrist-worn watch, a chest strap, a smart garment, medical ear buds/over the ear, a forehead or nose sensor, a smart ring, or so forth.

The illustrative wearable CBT sensor 10 includes a thermoresistant structure 12. While FIG. 1 shows two layers of a thermoresistant structures 12, the CBT sensor 10 can comprise a single layer or any other suitable number of layers. In some embodiments, the thermoresistant structure 12 can comprise foam, or any other suitable thermoresistant material having a high resistance so that the thermoresistant structure 12 can be made from small layers of material. As shown in FIG. 1, the thermoresistant structure 12 has a proximal surface 11 configured for attachment to skin S of the associated patient, and a distal end 13 opposite from the proximal surface 11. A contact interface between the proximal surface 11 of the thermoresistant structure 12 and the skin S comprises an adhesive 9 adhering the proximal surface 11 to the skin S.

The CBT sensor 10 also includes a plurality of temperature sensors 14 arranged to measure temperatures of the thermoresistant structure 12 at a corresponding plurality of different distances from the proximal surface 11 of the thermoresistant structure 12. The thermoresistant structure 12 comprises a plurality of thermoresistant layers 12a, 12b bonded together and the temperature sensors 14 are disposed at bonding interfaces between neighboring thermoresistant layers 12a and 12b. For example, as shown in FIG. 1, the plurality of temperature sensors 14 include a proximal temperature sensor 14a arranged to measure a temperature of the thermoresistant structure 12 at the proximal surface 11, a distal temperature sensor 14b arranged to measure a temperature of the thermoresistant structure 12 at the distal end 13, and at least one intermediate-position temperature sensor 14c arranged to measure a temperature of the thermoresistant structure 12 at a location between the proximal surface 11 and the distal end 13 (and in the illustrative example, specifically at the interface between the thermoresistant layer 12a and the thermoresistant layer 12b). This provides three temperature measurements acquired by the three respective temperature sensors 14a, 14b, and 14c for extrapolating the core body temperature.

More generally, it will be appreciated that if the thermoresistant structure 12 includes N thermoresistant layers (illustrative N=2 in FIG. 1) then there can be N+1 temperature sensors arranged at the proximal surface 11, distal surface 13, and at each interface between two adjacent thermoresistant layers of the N-layer stack. In general, increasing the value of N by increasing the number of layers increases the number of temperature measurements used for extrapolating the core body temperature, which may increase accuracy of the extrapolation. However, this is balanced by a desire to limit the overall thickness of the thermoresistant structure which biases toward a smaller value for N. Viewed alternatively, for a given overall thickness the thermoresistant structure, increasing the number of layers N entails decreasing the thickness of each layer which can adversely affect accuracy of the core body temperature measurement. Hence, the number of layers N is suitably chosen based on specific design considerations such as the intended thickness of the thermoresistant structure 12 and the thermal resistivity of the material making up the thermoresistant layers. Moreover, while the illustrative thermoresistant structure 12 is made up of a stack of thermoresistant layers 12a, 12b, in other embodiments the thermoresistant structure could be a single unit, e.g. a single layer or block of thermoresistant material, with the temperature sensor(s) 14c embedded in that single layer or block of thermoresistant material.

As previously discussed, the accuracy of the extrapolated CBT measurement depends on the temperature difference between the temperature measured by the proximate temperature sensor 14a and the distal temperature sensor 14b. If this temperature difference is too small, then accuracy of the CBT measurement can be compromised.

To enhance CBT measurement accuracy, the CBT sensor 10 also includes a temperature-changing element 16 configured to change a temperature of the distal end 13 of the thermoresistant structure 12. This provides for independent control of the temperature of the distal end 13 measured by the distal temperature sensor 14b. As shown in FIG. 1, the temperature-changing element 16 is disposed in a cover 18 disposed over the distal end 13 of the thermoresistant structure 12. This has the advantage that the cover 18 can prevent clothing or the like from coming into direct contact with the temperature-changing element 16. In one embodiment, the temperature-changing element 16 comprises one or more heating elements, such as a resistive heating element, configured to heat the cover 18. In another embodiment, the temperature-changing element 16 comprises one or more cooling elements (for example, Peltier elements) configured to cool the distal end 13 of the thermoresistant structure 12.

The CBT sensor 10 also includes an electronic processor 20 disposed in an electronics module (although not shown, it will be appreciated that the electronics module also includes an on-board battery or other on-board electrical power source to power the temperature-changing element 16 and the electronics processor 20). The electronic processor 20 is programmed to acquire temperature measurements from the plurality of temperature sensors 14 while operating the temperature-changing element 16 to maintain a temperature of the distal end 13 of the thermoresistant structure 12 at a temperature different from an ambient temperature where the patient is located. To do so, the electronic processor 20 is programmed to acquire the temperature measurements from the plurality of temperature sensors 14 while operating the one or more cooling elements 16 to maintain the distal end 13 of the thermoresistant structure 12 at a temperature below the ambient temperature. In another example, the electronic processor 20 is programmed to acquire the temperature measurements from the plurality of temperature sensors 14 while operating the one or more heating elements 16 to maintain the distal end 13 of the thermoresistant structure 12 at a temperature above the ambient temperature.

In the just-mentioned examples, a closed-loop feedback control is suitably employed to maintain the temperature of the distal end 13 at a setpoint temperature using as feedback the temperature measured by the temperature sensor 14b. To do so, the processor 20 may suitably implement a proportional-integral-derivative (PID) temperature controller. However, in other embodiments, the electronics processor 20 may not actively control the temperature of the distal end 13, as it is sufficient for the temperature of the distal end 13 to be different enough from the temperature of the proximal surface 11 to provide a sufficient temperature gradient for accurate CBT extrapolation. Hence, in some embodiments the temperature-changing element 16 may operate in an open-loop fashion, without feedback control by the electronic processor 20.

From the acquired temperature measurements, the electronic processor 20 is programmed to extrapolate a core body temperature of the associated patient. To do so, the electronic processor 20 is programmed to perform linear extrapolation to extrapolate the core body temperature of the associated patient from the acquired temperature measurements. (If the number of temperature measurements is sufficiently large, e.g. if the number of layers N is large, then polynomial, nonlinear extrapolation, or any other prediction method, is alternatively contemplated). In doing so, the noise level of the acquired temperature measurements is reduced, which increases the accuracy of the measurements. In addition, the thickness of the thermoresistant structure 12 can be reduced, resulting in more convenience for the patient. The core body temperature of the patient is then output on a display device 22.

With continuing reference to FIG. 1, the CBT sensor 10 can be employed by itself, or could be integrated with additional health monitoring. For example, the CBT sensor 10 could be incorporated into an ambulatory adhesive health monitoring patch configured to measure heart rate (HR) and/or other vital signs, such as the Philips MCOT® (mobile Cardiac Telemetry) ECG monitor. The nonlimiting illustrative example of the Philips MCOT® monitor includes electrodes for ECG monitoring of the heart rate, and the patch communicates with a pocketable monitor with wireless connectivity (e.g. 3G/4G/5G cellular connectivity) to enable real-time or near real-time transmission of HR data to a hospital or other health monitoring center. By adding the CBT sensor 10 to such a device, both HR and CBT measurements can be acquired and the patient's health can thereby be more accurately assessed.

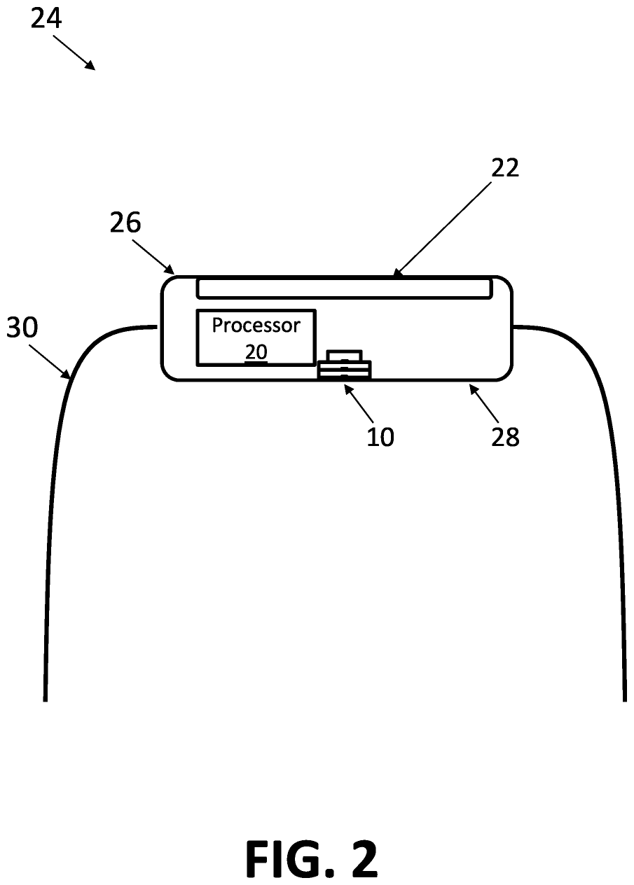

With reference to FIG. 2, in some embodiments, a wristwatch 24 can include a watch 26 having a front face with the display device 22, and a back face 28 configured to contact the skin S. A wristband 30 holds the wristwatch 24 on the wearer's wrist. When worn on the wrist, it will be appreciated that the back face 28 will be in contact with skin of the wrist. The wristwatch 24 can include the CBT sensor 10 disposed on the back face 28 with the proximal surface 11 of the CBT sensor 10 arranged to contact the skin of the associated patient when the associated patient wears the wristwatch 24. In this case, the pressure of the wristband 30 holding the watch on the wrist provides the contact of the proximal surface 11 of the CBT sensor 10 to the skin, so that the adhesive 9 shown in FIG. 1 is typically omitted in this embodiment.

As previously discussed, another source of CBT measurement error is failure to account for the temperature drop (or rise) across the interface between the skin S and the proximal surface 11 of the CBT sensor 10. This temperature drop (or rise) is a consequence of the nonzero thermal resistance of the interface. In the example of FIG. 1, the adhesive 9 may contribute to this interface thermal resistance. In the wristwatch example of FIG. 2, air gaps or pockets between the proximal surface 11 of the CBT sensor 10 and the skin can contribute to the interface thermal resistance. In some embodiments, the electronic processor 20 is programmed to calibrate the core body temperature sensor 10 to compensate for this interface thermal resistance by performing a calibration method 100.

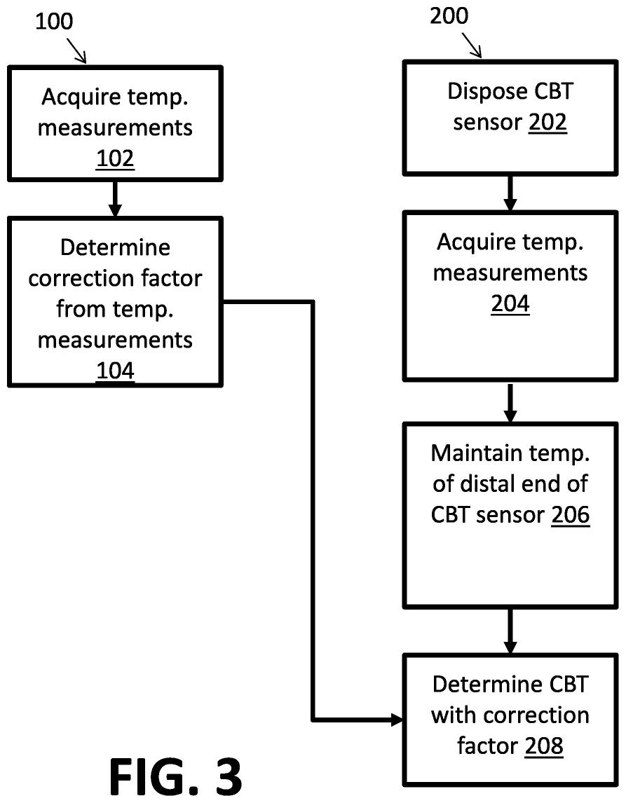

With reference now to FIG. 3, and with continuing reference to FIGS. 1 and 2, an illustrative embodiment of the calibration method 100 is shown by way of a flowchart. At an operation 102, the electronic processor 20 is programmed to control the temperature-changing element 16 to acquire temperature measurements from the plurality of temperature sensors 14 for a plurality of different temperatures of the distal end of the thermoresistant structure 12. For example, the proximal temperature sensor 14a measures a temperature of the thermoresistant structure 12 at the proximal surface 11, the distal temperature sensor 14b measures a temperature of the thermoresistant structure 12 at the distal end 13, and the intermediate-position temperature sensor 14c measures a temperature of the thermoresistant structure 12 at a location between the proximal surface 11 and the distal end 13. At an operation 104, a correction factor that corrects for a temperature drop across the interface 9 between the proximal surface 11 of the thermoresistant structure 12 and the skin S of the associated patient is determined.

To perform the calibration method 100, a (thermal) resistance between the proximal temperature sensor 14a and the skin S is determined, which depends on a thermal coupling between the thermoresistant structure 12 and the skin S. For example, the electronic processor 20 is programmed to solve a series of Equations (1)-(3):

$$C B T - {T}_{14 a} = ( {R}_{contact} / {R}_{total} ) * {T}_{c o v e r} - CBT ) \qquad ( 1 )$$

where CBT is a CBT measurement made by the CBT sensor, T₁₄ₐ is a temperature of the thermoresistant structure 12 measured by the proximal temperature sensor 14a at the proximal surface 11, R_{contact} is a resistance between the proximal temperature sensor 14a and the skin S, Rₜₒₜₐₗ is a total resistance of the CBT sensor 10, and T_{cover} is a temperature of the cover 18.

$$C B T - {T}_{14 c} = ( ( {R}_{contact} + {R}_{Foam 12 a} ) / {R}_{total} ) * ( {T}_{cover} - CBT ) = ( CBT - {T}_{14 a} ) + ( {R}_{F o a m 1} / {R}_{total} ) * ( {T}_{c o v e r} - CBT ) \qquad ( 2 )$$

where T_{14c} is a temperature of the thermoresistant structure 12 measured by the intermediate-position temperature sensor 14c at a location between the proximal surface 11 and the distal end 13, and R_{Foam1} is a resistance of the first layer 12a of the thermoresistant structure 12.

$${T}_{14 a} - {T}_{14 c} = ( {R}_{F o a m 12 a} / {R}_{total} ) * ( {T}_{c o v e r} - CBT ) \qquad ( 3 )$$

In lieu of temperature of the cover 18 (T_{cover}), a temperature of the thermoresistant structure 12 measured by the distal temperature sensor 14b at the distal end 13 can be used. By varying T_{cover}, and assuming CBT stays constant during that measurement period, and measuring T₁₄ₐ-T_{14c}, the ratio (R_{Foam1}/Rₜₒₜₐₗ) can be determined, using Equation (4):

$$1 / α = ( {R}_{Foam 12 a} / {R}_{total} ) = Δ ( {T}_{14 a} - {T}_{14 c} ) / Δ ( {T}_{cover} ) \qquad ( 4 )$$

By substituting α in Eq. 4 (defined in first part of Equation (4) into Equation (3), Equation (5) follows. α is determined by second part of Equation (4), viz by varying T_{cover} and observing change in T₁₄ₐ-T_{14c}, CBT can be computed for a given T_{cover}, T₁₄ₐ, and T_{14c} according to Equation 5:

$$CBT = {T}_{cover} - α * ( {T}_{14 a} - {T}_{14 c} ) \qquad ( 5 )$$

Once the calibration method 100, is complete, the electronic processor 20 is programmed to calibrate perform a CBT measurement method 200. With continuing reference to FIG. 3, an illustrative embodiment of the CBT measurement method 200 is shown by way of a flowchart. To begin the method 200, at an operation 202, the proximal surface 11 of the thermoresistant structure 12 is disposed in thermal contact with the skin S of the patient.

At an operation 204, temperatures of the thermoresistant structure 12 are measured at a plurality of different distances from the proximal surface 11 of the thermoresistant structure 12. For example, the proximal temperature sensor 14a measures a temperature of the thermoresistant structure 12 at the proximal surface 11, the distal temperature sensor 14b measures a temperature of the thermoresistant structure 12 at the distal end 13, and the intermediate-position temperature sensor 14c measures a temperature of the thermoresistant structure 12 at a location between the proximal surface 11 and the distal end 13.

At an operation 206, during the measuring of the temperatures, a temperature of the distal end 13 of the thermoresistant structure 12 is maintained at a temperature different from an ambient temperature by controlling the temperature-changing element 16 to heat or cool the cover 18.

At an operation 208, a CBT of the patient is extrapolated from the measured temperatures. To do so, the electronic processor 20 is programmed to applying the correction factor from the calibration method 100 to correct for the temperature drop over the interface 9 between the proximal surface 11 of the thermoresistant structure 12 and the skin S of the patient. The measured CBT of the patient is displayed on the display device 22.

The disclosure has been described with reference to the preferred embodiments. Modifications and alterations may occur to others upon reading and understanding the preceding detailed description. It is intended that the exemplary embodiment be construed as including all such modifications and alterations insofar as they come within the scope of the appended claims or the equivalents thereof.

1. A core body temperature sensor comprising:
a thermoresistant structure having a proximal surface configured for attachment to skin of an associated patient, and a distal end opposite from the proximal surface;
a plurality of temperature sensors arranged to measure temperatures of the thermoresistant structure at a corresponding plurality of different distances from the proximal surface of the thermoresistant structure;
a temperature-changing element configured to change a temperature of the distal end of the thermoresistant structure; and
an electronic processor programmed to:
acquire temperature measurements from the plurality of temperature sensors while operating the temperature-changing element to maintain a temperature of the distal end of the thermoresistant structure at a temperature different from an ambient temperature, and
extrapolate a core body temperature of the associated patient from the acquired temperature measurements.

2. The core body temperature sensor of claim 1, wherein the plurality of temperature sensors include:
a proximal temperature sensor arranged to measure a temperature of the thermoresistant structure at the proximal surface;
a distal temperature sensor arranged to measure a temperature of the thermoresistant structure at the distal end; and
at least one intermediate-position temperature sensor arranged to measure a temperature of the thermoresistant structure at a location between the proximal surface and the distal end.

3. The core body temperature sensor of claim 1, wherein the thermoresistant structure comprises a plurality of thermoresistant layers bonded together and the temperature sensors of the plurality of temperature sensors are disposed at bonding interfaces between neighboring thermoresistant layers.

4. The core body temperature sensor of claim 1, wherein the thermoresistant structure comprises foam.

5. The core body temperature sensor of claim 1, wherein:
the temperature-changing element comprises one or more cooling elements configured to cool the distal end of the thermoresistant structure; and
the electronic processor is programmed to acquire the temperature measurements from the plurality of temperature sensors while operating the one or more cooling elements to maintain the distal end of the thermoresistant structure at a temperature below the ambient temperature.

6. The core body temperature sensor of claim 5, wherein the one or more cooling elements comprise Peltier elements.

7. The core body temperature sensor of claim 1, wherein the temperature-changing element comprises one or more heating elements configured to heat a cover.

8. The core body temperature sensor of claim 1, wherein the electronic processor is programmed to perform linear extrapolation to extrapolate the core body temperature of the associated patient from the acquired temperature measurements.

9. The core body temperature sensor of claim 8, wherein the electronic processor is further programmed to calibrate the core body temperature sensor by performing a calibration method comprising:
controlling the temperature-changing element to acquire temperature measurements from the plurality of temperature sensors for a plurality of different temperatures of the distal end of the thermoresistant structure; and
determining a correction factor that corrects for a temperature drop across an interface between the proximal surface of the thermoresistant structure and the skin of the associated patient.

10. The core body temperature sensor of claim 9, wherein the contact interface between the proximal surface of the thermoresistant structure and the skin of the associated patient comprises an adhesive adhering the proximal surface to the skin.

11. The core body temperature sensor of claim 1 further comprising:
a cover disposed over the distal end of the thermoresistant structure.

12. A wristwatch, comprising:
a watch having a front face with a display and a back face; and
a core body temperature sensor as set forth in claim 1 disposed at the back face with the proximal surface of the core body temperature sensor arranged to contact skin of the associated patient when the associated patient wears the wristwatch.

13. A core body temperature measurement method, comprising:
disposing a proximal surface of a thermoresistant structure in thermal contact with skin of a patient;
measuring temperatures of the thermoresistant structure at a plurality of different distances from the proximal surface of the thermoresistant structure;
during the measuring of the temperatures, maintaining a temperature of a distal end of the thermoresistant structure at a temperature different from an ambient temperature using a heating or cooling element; and
using an electronic processor, extrapolating a core body temperature of the patient from the measured temperatures.

14. The core body temperature measurement method of claim 13, wherein the maintaining comprises:
during the measuring of the temperatures, maintaining the temperature of the distal end of the thermoresistant structure at a temperature which is below the ambient temperature using a cooling element.

15. The core body temperature measurement method of claim 13, further comprising:
determining a correction factor for a temperature drop over an interface between the proximal surface of the thermoresistant structure and the skin of the patient based on temperatures of the thermoresistant structure measured at the plurality of different distances from the proximal surface of the thermoresistant structure for different temperatures of the distal end of the thermoresistant structure set using the heating or cooling element;
wherein the extrapolation of the core body temperature includes applying the correction factor to correct for the temperature drop over the interface between the proximal surface of the thermoresistant structure and the skin of the patient.
